(19) United States
(12) Patent Application Publication
(10) Pub. No.: US 2026/0194478 A1
(43) Pub. Date: Jul. 9, 2026

(54) SURFACE MASS SPECTROMETRY DEVICE

(71) Applicant: Peter Jonathan Cumpson, Sydney (AU)

(72) Inventor: Peter Jonathan Cumpson, Sydney (AU)

(21) Appl. No.: 19/132,802

(22) PCT Filed: Dec. 13, 2023
(86) PCT No.: PCT/AU2023/051297

§ 371 Date: May 24, 2025

(30) Foreign Application Priority Data
Dec. 13, 2022 (AU) 2022903809
Nov. 30, 2023 (AU) 2023903879

(51) Int. Cl. G01N 23/2258; G16C 20/70

(52) U.S. Cl. CPC G01N 23/2258; G16C 20/70

(57) ABSTRACT

The present invention introduces an innovative Surface Mass Spectrometry Device designed to enhance the interpretability of Secondary Ion Mass Spectrometry (SIMS) spectra, especially for organic compounds. This device uniquely utilizes ultraviolet light and/or ozone and/or hydrogen to chemically modify the sample surface, thereby facilitating a more accurate analysis of the chemical moieties present. The method involves comparing SIMS spectra recorded before and after exposure to these modifying agents. Additionally, the invention includes a computational element that employs algorithms like fragmentation modelling and neural network deep learning for data processing. This approach promises to significantly improve the reliability and efficiency of SIMS analysis when obtaining structure information from SIMS spectra, reducing the need for extensive expert interpretation and making the technology more accessible and cost-effective.

### FIELD OF THE INVENTION

The present invention relates generally to the field of computational chemistry and machine learning in analytical chemistry. More specifically, it concerns a method for interpreting secondary ion mass spectrometry (SIMS) spectra of organic compounds using UV light, and/or ozone and/or hydrogen and data processing optionally using neural networks.

### BACKGROUND

In analytical laboratories there is a frequent need to characterise small samples chemically.

Techniques such as x-ray photoelectron spectroscopy (XPS) and secondary ion mass spectrometry (SIMS)^{1,2,3} can provide an excellent chemical characterization of the top few nanometers of the surface. Secondary ion mass spectrometry (SIMS) and static secondary ion mass spectrometry (SSIMS) are powerful tools for the analysis of complex molecules at the outermost surface (approximately 1 nm) of a solid with femtomole sensitivity, molecular specificity, sub-200 nm spatial resolution and excellent repeatability of better than 1% in the best cases. SIMS (or ion microprobing) is an extremely sensitive mass spectrometric technique that determines the elemental, isotopic, or molecular composition of a solid sample surface. SIMS uses an energetic primary ion beam generated by an ion gun to produce and expel under high vacuum secondary particles (primarily atoms or molecules depending on the SIMS mode) from the sample surface. These particles can be neutral or positively or negatively charged. Charged particles of one polarity are extracted from the sputtering area by an electric field. These secondary ions are then focused by an extraction lens and the secondary ion beam is analyzed by mass spectrometry, as shown schematically in FIG. 1. Although virtually all elements can be analyzed by SIMS, different elements possess different secondary ion yields and consequently their SIMS detection limit varies dramatically. SIMS is applied in two different modes to bombard the surface of the sample. Dynamic SIMS uses a continuous ion beam at a high dose so that the number of incident ions is higher than the number of surface atoms of the sample.

Static SIMS uses a very low total primary ion dose, so that the probability of any impact site being hit a by more than one ion is very low. As a result, the spectra one obtains in SSIMS are characteristic of the initial sample condition, not the effects of previous sputtering.

SSIMS is essential for analysis in a wide range of technologies from the interactions of proteins at surfaces and polymer interfacial behaviour to drug delivery systems and organic electronics. A major use of SSIMS is the identification of organics at surfaces. However, the complexity of SSIMS spectra and the difficulties in interpretation and identification have been significant barriers to the wider uptake of the technique

In SIMS one is essentially performing mass-analysis of fragments ejected from a damaged surface. In some other mass spectrometries one has much more control over the fragmentation and especially the ionization processes, so that one can more easily make sense of the mass spectra to deduce information about the chemical formula of the analyte. In SIMS though, one is limited to looking at the products of an uncontrolled energetic impact of a primary ion at the surface. For example, FIG. 2 shows a mass spectrum of morphine. It takes an expert to deduce the structure shown from this set of spike-shaped peaks. Ozonolysis can help in other mass spectrometries by making it easier to positively identify some moieties^{4,5,6}. It may be expected to be equally useful in SIMS, provided a simple way of exposing samples to ozone can be found. This is set out below.

In fact, the real situation is worse (in terms of the complexity of interpretation) than even FIG. 2 shows, because in static Secondary Ion Mass Spectrometry the relative secondary ion yields are strongly influenced by the primary ion mass and energy.

Different hardware options and new engineering developments have provided a wide range of ion sources and energies for analysts to use. The complexity of modern instruments is evidenced by the drawing of such an instrument in FIG. 3. Gallium and similar sources give high spatial resolution, caesium and oxygen enhance the negative and positive ion yields, respectively, and argon and xenon are the traditional sources for ultra-high vacuum studies. All these different conditions lead to different fragmentation patterns and different relative intensities for the peaks that do appear in the SIMS spectrum. As a consequence, the data reported from different laboratories may be expected to be significantly different and data in handbooks and libraries are only broadly comparable. This has been a major barrier to the wider use of SIMS; the interpretation expertise needed is rare and expensive (years of study) which reduces the market for SIMS instruments, which makes them more expensive per unit, which means the numbers remain small and few people choose to specialize in the technique—it's a viscous circle. Ironically, as the speed of commercial SIMS systems has increased, more spectra are generated each year, but there are only a limited number of skilled personnel to interpret them. The costs of SIMS analysis are shifting from the cost of instrument time to the cost of interpretation. One of the features of the invention described here is that (by generating more spectra that remove the ambiguities that would exist if only one were recorded) it uses extra instrument time (which is cheap) to reduce the analyst's time spent on interpretation (which is expensive) and improves reliability of the result and improves the ability to demonstrate that the correct conclusions have been reached to readers of analytical reports and scientific publications.

Looking at the SIMS literature as a whole, one way to address this complexity has been to look at a sequence of spectra under progressively different conditions rather than try to fully interpret a single SIMS spectrum. The invention described below also takes this approach, but first we should examine some of the approaches along similar lines that have already appeared in the literature.

### 1. Heavy Metal Labelling

In some cases, it is possible to label particular chosen chemical moieties with heavy metal elements that show up clearly in SIMS spectra^{7,8,9}. It can take a lot of effort to find the right conjugation chemistry for a particular species (often a PhD topic in itself). It can also be problematic as it requires wet chemistry to introduce these metals to the surface, and it can introduce other contaminations this way (to which SIMS is extremely sensitive).

### 2. Temperature and Ion Dose

Some researchers have looked at SIMS spectra as a function of temperature¹⁰ or ion dose¹¹, both typically introducing increased surface damage. Usually though, the motivation has been to elucidate the mechanism of damage, not to find out more about the chemistry initially present.

### 3. G-SIMS

Another type of “controllable damage” comes from the primary ion beam itself. G-SIMS¹² or “Gentle SIMS” is a type of SSIMS that requires the acquisition of at least two SSIMS mass spectra, one using an ion beam that gives high fragmentation conditions, and one using an ion beam to give low fragmentation conditions, potentially with intermediate cases too. Essentially both spectra, or the sequence of spectra, represent a sequence of increasing surface damage. One can either extrapolate back to zero primary ion energy to get an approximation of spectra that one would get from and undamaged surface or use the sequence¹³ to gain more information about (for example) which functional groups are present at the surface¹⁴. Initially in the literature this was done by taking spectra for at least two different energies of primary ion one after the other, but more effective is to use primary ions of very different mass. Indeed, at one stage hardware was developed (known as the “G-tip”¹⁵) to allow different primary ions to be used more easily¹⁶.

New Method: UV and/or Ozone and/or Hydrogen

Clearly, interpreting a series of spectra (by adjusting some parameter on which they depend) can often be simpler than trying to interpret a single spectrum. Many instrumental factors will be common to the sequence of spectra. Therefore, some kind of “internal” reference or method would be very useful. In other words, some way of altering the relative intensities of the different mass peaks away from their nominal values, and perhaps even functionalizing some of the functional groups present so that they appear at different mass values. This invention achieves this by exposure of the surface, in steps, to UV light and/or ozone gas (or UV light and/or hydrogen gas), producing oxidizing (and reducing) environments respectively where energy from UV promotes rapid surface oxidation (or reduction). One could view this as a way of introducing damage to the surface in controllable steps, which has some similarities to introducing variable degrees of damage in G-SIMS. However, it is much cheaper in the long term to use the highly-engineered ion gun as little as possible; a few UV lamps (examples of which are shown in FIG. 4) in an entry lock is simpler and cheaper to make, and much easier to repair or replace once some wear-and-tear has taken place. FIG. 5 shows a typical sample holder of the type that would be exposed to UV and/or ozone and/or hydrogen in this way. FIG. 6 shows a schematic diagram of how such a system may be controlled by the computer or programmable logic controller (PLC) operating the instrument.

Note that much research has been done on the practical effects of UV and ozone, but not for the purpose of interpreting the initial SIMS spectrum. There are lots of papers investigating what UV and/or ozone does to particular materials, for example to increase wettability for easier printing, or to investigate the effects of weathering on PVC window frames. In other words, the effect of UV and/or ozone when the initial material(s) are known from the beginning and the scientific question is: “what does UV and/or ozone do to this known starting material?”. This does not concern us here. We address a different problem; working out what the original material was when it is initially unknown. We are not interested in the effects of UV and/or ozone, except in so far as these changes can be used to infer information about the original, unknown material before UV and/or ozone exposure.

### DESCRIPTION OF THE INVENTION

This invention increases the interpretability of SIMS spectra by chemically modifying the surface being analysed using ultraviolet light and/or ozone in the presence of oxygen-containing gas (e.g. laboratory air) creating an oxidizing environment, or ultraviolet light and/or a gas (e.g. hydrogen) creating a reducing environment, thereby changing the proportions of the different chemical moieties at the surface, for example by increasing the proportion of highly oxidized states, or by exposure to a reducing atmosphere (e.g. hydrogen) in UV light for the purposes of the opposite; reduction. By comparing SIMS spectra recorded before and after this step (and optionally more than one such oxidative/reducing step) it extracts the component peaks in the mass spectrum numerically in a computer, for example using multivariate statistical methods (in some embodiments Principal Component Analysis (PCA), non-negative PCA or Singular Value Decomposition (SVD)) or neural network, deep learning or techniques similar to those that have been successful in Alphafold¹⁷. This UV/ozone exposure/SIMS spectrum acquisition cycle is done over a short time period and using the same SIMS settings (such as sample height), so that drift of the SIMS mass or intensity scale are both negligible.

The invention comprises;
1. An enclosure composed of materials that UV, hydrogen and ozone do not easily attack (e.g. metals, glass). It has a door or lid that is easy to open and close, and is largely (though not necessarily completely) airtight when the door or lid is closed. The door or lid, when open, permits the insertion of a sample holder.
2. A sample holder, in some embodiments of the type designed to hold common sample stub types used in electron microscopy and surface analysis (XPS, SIMS).
3. Within the said enclosure are one or more sources of UV light, preferably at least one of which is capable of emitting significant radiation at a sufficiently short wavelength to produce ozone in air at room temperature and pressure. In some embodiments these are mercury vapour lamps¹⁸, and in others short wave light emitting diodes (LEDs), or a combination of the two.
4. An electronic circuit that switches on the light source(s) for a predetermined time, or until a predetermined exposure of the sample holder to UV and/or ozone has been reached;
5. The ozone and/or UV produced within the enclosure are at sufficient levels to chemically modify the specimen surface, so that the chemical moieties seen in SIMS spectra are changed by this exposure, but sufficiently low that one still sees a progression of SIMS spectra after successive exposure steps rather than sudden radical changes that lead to the sudden loss of one mass peak or another. Within the said sample enclosure, or outside the sample enclosure but directed into it through an optical window, are one or more sources of UV light, at least one of which is capable of emitting significant radiation at a sufficiently short wavelength to assist in photo-catalytically aiding the reduction of sample surfaces in the presence of hydrogen gas. In some embodiments these are mercury vapour lamps¹⁹, or other kinds of discharge lamps such as xenon lamps, and in others short wave light emitting diodes (LEDs), or a combination of the these.
6. Reduction in a reducing atmosphere (e.g. hydrogen) and/or UV light may be performed as follows; oxygen is removed from the sample enclosure, optionally by pumping the air out of the sample enclosure to reach pressures in the range below 10⁻³ millibar, and preferably below 10⁻⁶ millibar. Alternatively, the sample enclosure may be purged with an inert gas such as nitrogen or argon. Hydrogen gas is introduced into the sample enclosure, optionally from a hydrogen gas generating cell or cells as described below, or optionally from an external hydrogen cylinder.
7. Optionally the zinc-air (or similar) battery may be fixed within a hydrogen-permeable cell-enclosure²⁰ (for example a palladium or palladium alloy tube) that allows hydrogen to leave the battery and pass through the wall(s) of the hydrogen-permeable cell-enclosure but prevents other species (such as water vapour) from doing so. Optionally, said permeable cell-enclosure may be heated²¹, for example by passing an electric current though it leading to cause Joule-heating, in order to increase the rate of hydrogen diffusion through its walls out into the main space within the sample enclosure containing the sample(s). Instead of a hydrogen-permeable cell-enclosure an impermeable one and pressure relief valve arrangement may be used as described below.
8. Optionally an electronic circuit that switches on a current through the said zinc-air or similar metal-air battery for a predetermined time, or pre-determined total charge passed or to a pre-determined hydrogen concentration, or until a predetermined exposure of the sample holder to UV and/or hydrogen has been reached;
9. The hydrogen and/or UV produced within the sample enclosure are at sufficient levels to chemically modify the specimen surface, so that the envelope of chemical states seen in SIMS spectra is changed by this exposure, as more reduced chemical states become more common at the surface.
10. Optionally sensors for the measurement of UV and/or hydrogen concentration within the said sample enclosure. These allow the UV and hydrogen levels to be reported to the user, so that repeatable and reproducible exposure of specimens to UV and hydrogen are possible, in some embodiments even under closed-loop (e.g. proportional-integral-derivative PID) control.
11. Optionally sensors for the measurement of UV and/or ozone concentration within the said enclosure. These allow the UV and ozone levels to be reported to the user, so that repeatable and reproducible exposure of specimens to UV and ozone are possible.
12. A Secondary Ion Mass Spectrometry (SIMS) instrument capable of recording high-resolution mass spectra of the type normally used for Static SIMS.
13. Computer processing of the resulting SIMS spectra to identify the components of the spectra.

The said enclosure can be entirely separate from the vacuum system, or form part of it (e.g. the entry-lock of the SIMS system, so that the sample block never leaves the automated sample handling system of the SIMS instrument). Ultraviolet (UV) and ozone production is achieved using small mercury lamp(s) or other discharge lamps or shortwave LEDs. Optionally the ozone is augmented from an external electrical ozone generator, such as a corona discharge type²². Ozone is produced in-situ from diatomic oxygen in air by illumination with very short wavelength UV light, in one embodiment 185 nm radiation from a mercury vapour lamp. Destruction of the ozone to allow the container to be opened is achieved by illuminating the contained air with longer wavelength UV, in one embodiment 254 nm radiation from a mercury vapour lamp (where the shorter 185 nm emission has been blocked by a glass envelope or filter). FIG. 6 shows schematically one embodiment of this part of the invention.

In FIG. 6 a battery or mains power supply (660) supplies energy to one of two UV lamps (640) labelled A and B. Both lamps, in this embodiment, are mercury vapour lamps. Both emit energy in the UV at both 185 nm and 254 nm. Lamp B has an optical filter (610) covering it so that only the longer of these two wavelengths reaches the air around the sample. A Programmable timer controls which lamp (if either) receives power. In each case a “ballast” component (620) is required (as for most discharge lamps and fluorescent lamps) to manage the voltage and current of the lamp through an acceptable range as it begins to operate—often initially a high voltage is applied to establish the discharge then a lower voltage and current to maintain it. The programmable timer, in this embodiment, powers the lamp A (illuminating the sample with UV light and forming ozone around it) then switches off lamp A and switches on lamp B (from which only 254 nm radiation is able to reach the space around the sample, decomposing what ozone remains) then finally switches both lamps off. This ensures that all ozone is quickly removed from the enclosure so that the sample can quickly and safely be put into the SIMS analysis chamber. In some embodiments the sample is on a slowly rotating stage, so as to homogenise the exposure to UV and ozone.

UV/Ozone Cleaning Equipments: Why the Present Invention is Different to these, and how the Differences Arise from Different Purposes

UV/ozone cleaning equipment has been used for several decades, for example as popularised by the work of J R Vig²³, concentrating on the use of mercury vapour lamps. Low-pressure mercury lamps have two principal emissions in the UV, at 185 nm and 254 nm. The 185 nm UV line decomposes oxygen molecules and synthesizes ozone, O₃ in situ. The 254 nm UV line decomposes ozone and produces high energy O*(activated oxygen). These highly-oxidative species interact with carbonaceous contamination on a surface (indeed, anything on the surface that can be oxidised). Ultimately, in combination with direct UV exposure (interacting strongly with C═O moieties via Norrish type chemical processes) organic species are oxidised and/or degraded to volatile compounds, mostly CO₂, which diffuses away from the surface. This process is shown schematically in FIG. 7.

Commercial UV/ozone cleaners are high-power devices designed to remove all carbonaceous contamination as rapidly as possible. They usually have no need to measure and report UV or ozone levels, instead simply being designed to supply very high levels of both so as to remove contamination rapidly. If surface chemical modification, rather than complete removal of carbonaceous contamination is attempted (for example to clarify chemical origin of SIMS mass spectra) then it is too easy to go “too far” and remove it all, because of the design aims of the UV/ozone cleaner. There is a further difference in design between commercial UV/ozone cleaners and the present invention, motivated by the different purpose. Usually commercial UV/ozone cleaners are designed to be used with large objects such as silicon wafers of 200 mm diameter or more. What we need for the present purpose is less power, so that surface species are gradually oxidised and are modified in steps of a fraction of a monolayer of modification, perhaps over several increasingly aggressive oxidation steps. And a smaller sample space so that the device can be placed close to the SIMS sample entry lock, for rapid exposure and then returning the sample to the SIMS system vacuum with the minimum of exposure time to atmospheric contamination. Also, measurements of UV intensity and ozone concentration help in ensuring reproducibility of measurements in different locations, so built-in UV and ozone monitors are useful in this SIMS application—i.e. they are optional but very useful as part of this invention.

Having said all this, I have in the past successfully modified UV/ozone cleaning apparatus to generate spectra for samples UV/ozone exposed. Typically, this has been done by modifying the apparatus, disabling it in some ways (e.g. pressing the “emergency stop” button after a few seconds to avoid excessive UV/ozone exposure) or disassembly to extract components (e.g. the lamp) then putting those components in a different enclosure. Indeed, much of the work that led to the present invention was done by modifying commercial UV/ozone units to achieve a purpose for which they were not designed.

SIMS instruments have occasionally been equipped with UV sources over the years, but this has been to study the effect of UV on particular materials^{24,25,26}, not modify arbitrary ones for improved information. The aim of previous researchers has been not to modify chemical states at the surface for the purpose of identifying chemical states as described in the present invention, but to study particular chemical reactions that particular UV exposure induces in particular specimen materials, for example weathering of organic materials in air²⁷.

### POSSIBLE EMBODIMENTS AND CONFIGURATIONS

FIG. 8 shows a schematic vertical cross-section through a typical existing commercial SIMS instrument. There is an analysis chamber (1500) nominally at ultra-high vacuum (UHV), with magnetic sector analyser (1510). Pumps (1505) maintain vacuum in the various parts of the system. Valves (1520) are opened and closed to allow a sample into the analytical chamber from the entry lock (1525). A transfer arm (1535) is used to move the sample between said analysis chamber and said entry lock. When withdrawing a sample from the system the entry lock is brought back up to atmospheric pressure by admitting gas (typically nitrogen) from cylinder (1515). The said entry lock typically has a transparent glass window (1530).

FIG. 9 shows one embodiment of the present invention in which the SIMS system and UV and/or ozone and/or hydrogen exposure enclosure are separate but in close proximity. The sample is moved, in air, from SIMS system to said enclosure (1610) containing the UV/ozone producing lamps (1620), and back again after UV/ozone exposure. These transfers could be automated using a small air-side robot arm or similar, but are most likely to be done manually by the operator.

FIG. 10 shows another embodiment of the present invention, in which the enclosure containing UV/ozone producing lamps is integrated with the SIMS system entry lock. This requires a UV-transparent window on said entry lock instead of the usual glass window (1530) and the back-fill gas cylinder (1700) to contain oxygen or an oxygen-containing gas mixture (e.g. dry air) rather than pure nitrogen. UV passes through the said UV-transparent window and creates ozone within the entry lock itself.

When the sample is present in the entry lock it is therefore exposed to UV and/or ozone, and can be moved back into the analysis chamber for the next spectrum acquisition. This configuration makes best use of automated sample handling, lamp and valve control; it would be possible, for example, for the whole of the sequence to run as an automated sequence under computer control and without the need for a human operator to be present. It could run overnight, for example, making good use of instrument time that would otherwise be difficult to make good use of.

### Possible Embodiments and Configurations: Hydrogen-Releasing Element

Many laboratories that operate SIMS instruments have high-purity hydrogen gas available. Others do not. In any case, when dealing with large quantities of hydrogen gas the costs of implementing safety procedures are often high, even if the quantities actually used (as in this application) are very small.

Therefore, optionally, and in some embodiments, we make use of hydrogen gas created in-situ within the sample enclosure by zinc-air or similar cell(s). These may be commercially available zinc-air “button” cells, sold for devices such as hearing aids, that have replaced the mercury cells common twenty years ago. Indeed, variants of such cells have been made commercially-available for the purpose of hydrogen production.

Therefore, to provide a simple source of high-purity hydrogen gas, optionally the said zinc-air, or other metal-air type battery is located within the sample enclosure, with an external control over the current passing through that battery. When a resistive load is applied over zinc-air batteries without access to oxygen, they generate²⁸ hydrogen gas at a fairly controllable rate²⁹. In one embodiment this may be achieved by having an external switch and resistor in series across the battery, so that switching-on will cause a resistor-limited current to pass through the battery.

This type of zinc-air battery is known to evolve a small quantity of hydrogen gas, roughly in proportion to the total charge that has passed through it. This allows hydrogen gas to be delivered to the region around the surface being analysed to a partial pressure of around 10-3 mb or above, greater than the pressure of other reactive species (e.g. potentially oxidative species such as oxygen, water etc) in the sample enclosure.

Zinc-containing cells designed specifically for hydrogen production (such as those made³⁰ by the Varta company) may be used—these are really modified forms of zinc-air battery marketed as precision hydrogen generators. The key consideration is that only small amounts of hydrogen are needed in this application, filling the small volume of a sample enclosure at what can be much less than atmospheric pressure, so that a zinc-air cell that produces perhaps 150 cm³ over its lifetime is quite sufficient. Four such cells in a battery will be able to deliver 600 cm³ over their lifetime, probably enough for >500 reduction cycles of low-pressure H₂ sample exposure under UV light before needing to be replaced.

Table 1 shows a product overview for two battery cell products from the Varta company designed specifically to produce hydrogen.

TABLE 1
Characteristics of commercially-available hydrogen-producing cells.

| | | | Typical | Volume of | | | |
|---|---|---|---|---|---|---|---|
| Article | | No. of | Gas Output | Article | Diameter | Height | Weight |
| Designation | Article No. | Cells | (ml)* | (ml) | (mm) | (mm) | (g) |
| V 150 H₂MF | 24690 101 501 | 1 | 150 | 0.55 | 11.6 | 5.5 | 1.65 |
| 2/V 150 H₂MF SK BE** | 24690 302 501 | 2 | 300 | 1.2 | 11.9 | 11.2 | 3.4 |

Other cell assemblies on request, e.g. for −1 | hydrogen
*at 20° C., 1013 hPa
**Assembly: SK = Stack up, BE = Bare (No final sleeve)

The cells cannot be used unenclosed within a vacuum chamber (the sample enclosure) such as a SIMS instrument entry-lock, because they contain an aqueous electrolyte. This will evaporate under dry conditions, and a vacuum is a very dry environment. Therefore, the cells must be enclosed by a container (the cell enclosure) within the sample enclosure that allows hydrogen gas out when required, but retains at least the partial-pressure of water at the operating temperature of the SIMS instrument, e.g. about 18 mmHg at 20° C.

There are at least two possible embodiments that will achieve this cell enclosure;

Possible cell enclosure embodiment A; A sealed tube around the cell(s) made from a hydrogen permeable (but water impermeable) material, such as palladium or palladium alloy. FIG. 11 shows a schematic of this arrangement, an optional embodiment in which four such cells are formed into a battery within a sealed palladium (or palladium alloy) tube that allows hydrogen to permeate out of it. FIG. 11 shows schematically one possible embodiment A of the permeation hydrogen-releasing element of the invention, FIG. 11(a) when de-selected, and FIG. 11(b) when selected to operate and release hydrogen. Alternatively;

Possible cell enclosure embodiment B; A sealed tube around the cells connected to the main space of the entry-lock or other sample enclosure through a normally-closed pressure-relief valve that opens when the pressure inside (caused by hydrogen produced by the cell(s)) exceeds a predetermined value above the vapour-pressure of water at that temperature. For example, a spring-loaded relief valve set to open when the pressure inside the cell enclosure rises above 0.2 atm above the pressure outside, in the sample enclosure. Some water vapour will escape each time the valve opens, but in its normally-closed state, in the many hours between instances of use, the cell(s) will not dry out.

FIG. 12 shows a schematic of a simple embodiment type B of the cell enclosure. In FIG. 12 The prior chosen weight of the ball, 1020, in funnel, 1030 allows pressure within the cell enclosure 1010 to exceed that of the saturated vapour pressure of water at the operating temperature of the device (typically room temperature or slightly above) even though the sample enclosure pressure (not to be confused with the cell enclosure) may be a vacuum. This ensures that the cells, 720, do not dry out. Momentary pressing of the pushbutton discharges the capacitor, so that when the button is released current flows through the cells, generating hydrogen, until the capacitor is fully charged, releasing a fixed quantity of hydrogen predetermined by the choice of the capacitance value C. The pressure of hydrogen within the cell enclosure, 1010, being higher than the pressure within the sample enclosure around it then momentarily displaces the ball, 1020, releasing a small, fixed and predetermined quantity of pure hydrogen into the region around the sample in the sample enclosure for reduction, or UV-assisted reduction, of the sample surface.

FIG. 13 shows schematically a slightly more sophisticated possible embodiment of cell enclosure type B, in both (a) dormant and (b) hydrogen-producing states. Here the switch controlling hydrogen production is normally open, as shown in (a), and may be a relay or similar switch under the control of the programmable logic controller, 750. The pressure around the cells, 720, within the cell enclosure, 930, is higher than inside the sample enclosure (not to be confused with the cell enclosure, 930) as a result of the pressure-relief valve formed by spring, 960, adjustment screw, 970, and “poppet”, 960. When, as shown in (b), the PLC closes the switch, d.c. current (limited by the carefully-chosen resistor R to limit the rate of hydrogen production) passes through the cells, 720. The cells release hydrogen until this pressure on the poppet is enough to overcome the force of the spring, 960, and the hydrogen escapes into the sample enclosure. In this embodiment the computer-code being executed by the PLC may select different durations for the switch closure, thereby releasing different quantities of hydrogen into the region around the sample for subsequent reduction, or UV-assisted reduction, of the sample surface.

### Algorithms and Data Processing

This is a step which appears in the previous filing (Australian provisional patent application 2022903809) as “step 13” on page 14 of that filing, and indeed as “step 13” above in the “Description of the Invention” section.

In SIMS one is essentially performing mass-analysis of fragments ejected from a damaged surface. In some other mass spectrometries one has much more control over the fragmentation and especially the ionization processes, so that one can more easily make sense of the mass spectra to deduce information about the chemical formula of the analyte. In SIMS though, one is limited to looking at the products of an uncontrolled energetic impact of a primary ion at the surface. For example, FIG. 2 shows a mass spectrum of morphine. It takes an expert to deduce the structure shown from this forest of spike-shaped peaks. Many analytes have even more complex spectra in positive or negative ion mode—sometimes both. Polymers can give rise to secondary ions representing a whole range of different numbers of monomer units, modified in several different ways by the energetic impact. This leads to a forest of peaks that leaves newcomers to the technique not knowing where to start or which peak to try to assign to a structure first.

In a previous initial filing (Australian provisional patent application 2022903809), and in this one, I have described a method based on ozonolysis that can help—by making it easier to positively identify some moieties^{31,32,33}. It is good to remember that, prior to the use of modern spectroscopic techniques, ozonolysis was a major tool for analytical chemists investigating the structure of organic compounds. I propose that when a technique such as SIMS has some unavoidable drawbacks, the introduction of ozonolysis can help mitigate some of them.

Set out below is a new computational method for the interpretation of SIMS spectra in a more automated way, which may be applied with or without the UV and/or ozone and/or hydrogen, but which may be expected to be useful synergistically with them. Since the first filing (Australian provisional patent application 2022903809 of 13 Dec. 2022) I have used primarily ozone alone rather than UV and ozone together, to avoid sub-surface damage by UV light, but there are many specimens for which using both in conjunction will be advantageous.

In some spectroscopies excellent progress has been made in applying machine learning methods to spectra³⁴. In the SIMS context this would mean an automated algorithm for giving the structure of the analyte from its SIMS spectrum. This important step is currently done by chemical and physical reasoning by a human expert. This can be very lengthy and expensive, and without years of training can be error-prone too.

In fact, the real situation is worse (in terms of the complexity of interpretation) than even the forest of peaks in FIG. 2 shows, because in static Secondary Ion Mass Spectrometry the relative secondary ion yields are strongly influenced by the primary ion mass and energy. Different hardware options and new engineering developments have provided a wide range of ion sources and energies for analysts to use. Gallium and similar sources give high spatial resolution, cesium and oxygen enhance the negative and positive ion yields, respectively, and argon and xenon are the traditional sources for ultra-high vacuum studies. Over the last 10 years or so cluster-ion sources, for example argon clusters or even water clusters have become increasingly popular in the field. All these different conditions lead to different fragmentation patterns and different relative intensities for the peaks that appear in the SIMS spectrum. As a consequence, the data reported from different laboratories may be expected to be significantly different, and data in handbooks and libraries are only broadly comparable. This has been a major barrier to the wider use of SIMS; the interpretation expertise needed is rare and expensive (requiring years of study) which reduces the market for SIMS instruments, which makes them more expensive per problem solved, which means the numbers remain small and few people choose to specialize in the technique—it's a vicious circle. Ironically, as the speed of commercial SIMS systems has increased, more spectra are generated each year, but there are only a limited number of skilled personnel to interpret them. The costs of SIMS analysis are shifting from the cost of instrument time to the cost of interpretation. One of the features of the invention described here is that (by generating more spectra that remove the ambiguities that would exist if only one were recorded) it uses extra instrument time (which is cheap) to reduce the analyst's time spent on interpretation (which is expensive) and improves reliability of the result and improves the ability to demonstrate that the correct conclusions have been reached to readers of analytical reports and scientific publications.

### A Solution to Part of the Problem

Much work has been done in recent years on a general understanding of electrospray ionisation mass spectrometry (ESI-MS)³⁵ which has become an increasingly important technique in the clinical laboratory for structural study or quantitative measurement of metabolites in a complex liquid biological samples. Indeed, the more controlled nature of the ionization mechanism in ESI-MS makes it more amenable than SIMS to the automated simulation of the mass spectrum of a molecule. Notable advances include those of the Wishart lab at Pacific Northwest National Laboratory in the US³⁶. One of the ways in which ESI is simpler than SIMS is that there is a well-defined ionization energy. So, the software from the Wishart lab, for example, takes this energy as an input parameter and calculates the likely fragmentation spectrum from it. Even for a single, pure analyte, the ESI spectrum can look very different depending on this ionization energy. More ionization energy leads to more fragmentation and a spectrum that shows the peaks from more fragments of lower mass than the molecular ion. By comparison, the SIMS spectrum is the result of an impact by a primary ion, so the ionization energy is much less well-defined; very close to the impact the energy is high, leading to a high degree of fragmentation and small molecular fragments are detected from this region (often with little remaining information about the molecular identity of the surface). This is shown schematically in FIG. 14. Further from the impact site the energy received from the impact is lower, leading to less fragmentation and perhaps a peak in the SIMS spectrum corresponding to the molecular ion, or large fragment ions from it. Therefore, we can expect the SIMS spectrum to resemble somewhat the sum of ESI-MS spectra for a number of different ESI-MS ionization energies. However, there are other SIMS-specific surface processes going on too, and these (as we shall see) we accommodate using an autoencoder neural network. This is a computer neural network, and we use it to learn the differences between a simulated ESI-MS spectrum and the real SIMS spectrum for a series of known analytes, so that when presented with the SIMS spectrum from an unknown analyte (or mixture of unknown analytes) it can predict what the in silico generated ESI-MS spectrum should be. Matching that spectrum to find the unknown analyte is easier, and can be automated much more easily, than searching directly for matching library SIMS spectra acquired using other instruments.

The dependence of the details of the SIMS spectrum on the exact local conditions (instrument settings, matrix composition, primary ion type etc) means that some kind of “internal” reference or method would be very useful. In other words, some way of altering the relative intensities of the different mass peaks away from their nominal values, and perhaps even functionalizing some of the functional groups present so that they appear at different mass values. This invention achieves this by exposure of the surface, in steps, to UV light and/or ozone gas or UV light and/or hydrogen gas, producing oxidizing and reducing environments respectively, where energy from UV promotes rapid surface oxidation or reduction.

There are key aspects the numerical method:
Oxidation Model: We use an oxidation model, either rule-based or a trainable model, that predicts the changes in molecular structure given a certain degree of oxidative exposure. This model acts as a prior knowledge source that can inform about probable changes that molecules undergo upon oxidation.
Incorporating Temporal Order: Ensures proper account is taken of temporal order, i.e., later oxidation progresses monotonically as exposure to ozone (or ozone and UV) increases.

A solution to this issues is the workflow shown schematically in FIGS. 15 and 16. There is a distinct training phase (FIG. 15) and an application phase (FIG. 16) in the use of this arrangement.

### Training Phase

1. Choose a molecular representation of molecule m, e.g. SMILES^{37,38,39}, for the molecule in question (one for which the composition is known, and the SIMS spectrum can be recorded). The molecule m is one of a training set of compounds that may comprise N compounds, where N may be in the range of tens or hundreds.
2. Generate synthetic mass spectrum Sₘ(E₁) at “impact energy” E₁, using methods developed for ESI-MS (e.g. Wang et al⁴⁰)
3. Repeat step 1 for impact energies E₂, E₃, . . . Eₙ, giving spectra Sₘ(E₂), Sₘ(E₃), . . . Sₘ(Eₙ) (Typically n may be around 3 or 4 in order to capture fragmentation vs. energy behaviour).
4. Take the molecular representation of molecule m, for example in SMILES format, and generate new representations (e.g. in SMILES) by modifying the SMILES to represent the molecule after ozonolysis.
5. Repeat steps 2 and 3 for this new in silico “ozonolyised” molecule.
6. Repeat steps 4 and 5 a small number of times, between 1 and around 10, generating spectra representing increasing degrees of ozonolysis.
7. Acquire a set of experimental spectra for molecule m using Static Secondary Ion Mass Spectrometry (SSIMS) for progressively greater ozone and/or UV exposure. Ozone may be generated by UV lamp or electrostatic discharge and applied to the specimen in the entry lock, for example.
8. Model the relationship between the set of simulated spectra for m to the experimental spectra for m using a invertible mathematical model, such as a singular value decomposition (SVD) or, preferably, an autoencoder neural network.
9. Repeat steps 1 to 8 for N molecules m=1,2,3, . . . , N, where there are N molecules in the training library.

The function of training is for the autoencoder to learn the relationship between the simulated ESI-MS spectra for the progressively oxidized molecules and the real SIMS spectra for progressive ozone and/or UV oxidation. This will include some of the complex matrix and surface effects that can greatly affect ionization yield in SIMS. The complexity, and nonlinearity of these relationships makes a deep neural network autoencoder suitable here. Though this does not easily allow these relationships to be understood by the human operator, they generally do not need to be.

This process is shown schematically in FIG. 15. Note that there may be different numbers of oxidation steps in the SIMS experimental work compared to the in silico simulation. In FIG. 15 we show three in silico steps and four SIMS spectrum recording events, but in practice there are typically 1 to 10 of each, and not necessarily the same number of each.

### Application Phase

In the application phase we use the (now trained) autoencoder to help select a chemical structure M that fits the observed SIMS spectrum (of an initially unknown analyte) best. This process is illustrated in FIG. 16.

The autoencoder is fed the measured SIMS spectra (for a series of increasing UV and/or ozone and/or hydrogen exposures), generating approximate ESI-MS spectra for a sequence of increasing oxidation. These are then compared to the output of the in silico ESI-MS simulation software, and scored (typically by a sum-of-squared-residuals metric). In this way a goodness-of-fit metric can be calculated for candidate molecule M. Either the user specifies M (based on chemical knowledge of the likely chemical components of the surface) or M itself is a candidate generated by software, and the best fit chemical structure is presented to the user as the result of this “spectra-to-structure” analysis.

### Possible Embodiment

Here we sent out one possible embodiment of the hardware and software set out above derived from what has so far been explored experimentally.

Ozone exposure was in the range 0.1 to 10 ppm produced in laboratory air using a small (type GTL3) UV 10V lamp with an E17 fitting. These are commercially available from many suppliers but require care to ensure you have the ozone-producing type rather than the non-ozone producing type. Typical ozone concentrations were around 6 ppm. This could also have been generated using a small corona discharge unit⁴¹ without any UV at all. In our recent experiments the UV generated by the lamp has been blocked from reaching the specimen, but this may not always be advantageous. SIMS spectra were acquired on an Iontof IV SIMS instrument from the Iontof company (Muenster, Germany). The software comprised several components running on a Dell latitude E7590 computer;
1. ESI-MS simulation using the Docker⁴² image made available by the Wishart group at PNNL, CFM-ID 4.0, “Competitive Fragmentation Modeling and Metabolite Identification”⁴³. This ran under Microsoft Windows 10 home edition as a Linux container in Docker. We used the three different ionization (a.k.a fragmentation) energies that are the defaults within CFM-ID for spectrum simulations; E₁=10 eV, E₂=20 eV and E₃=40 eV.
2. The ESI-MS container was issued commands from an Octave (version 8.1.0) script running under Windows 10.
3. The Autoencoder was implemented in Julia using the Julia Flux framework, a library for machine learning. The autoencoder has two dense layers at its input from the ESI-MS simulations, two dense layers at the input from the experimental SIMS spectra, and a 30 element middle layer. These were combined into an autoencoder using the Flux “Chain” function.
4. Octave was used to implement the ozonolysis simulation steps by parsing the input SMILES string for unsaturated carbon-carbon bonds, i.e. “C═C” or “C#C”. There is enough moisture present to complete hydrolysis, so this means we replace these substrings with “C=O·OC” or “CO·O=C” (which of the two to substitute is chosen at random) and “C═O·O=C” respectively. The code in FIGS. 17 and 18 illustrate how this is done. In future a more sophisticated approach to this substitution including unsaturated ring structures will be implemented.

FIG. 19 shows some results of applying this procedure to the morphine molecule (FIG. 2 showed a SIMS spectrum of the same substance) calculated using CFM-ID 4.0, “Competitive Fragmentation Modeling and Metabolite Identification” as a linux container in Docker. Here there is space to show only a sample of these results. FIG. 19(a) shows the morphine molecule and its calculated ESI-MS [M+H]+ spectrum. In fact, three mass spectra are shown here for three different fragmentation energies; E₁=10 eV (x), E₂=20 eV (+) and E₃=40 eV (filled circle). In SMILES representation morphine has the structure: CN1CCC23C4C1CC5═C2C(═C(C=C5)O)OC3C(C=C4)O

FIGS. 19(b) and (c) show two different molecules that result from the in silico ozonolysis procedure of step 4 above, in which Octave code has replaced an unsaturated bond in the morphine molecule with two different potential products of ozonolysis; CN1CCC23C4C1CC5═C2C(═C(C═O·OC5)O)OC3C(C=C4)O and CN1CCC23C4C1CC5=C2C(═C(CO·O=C5)O)OC3C(C=C4)O respectively. Other products are available, and generated by the Octave code, but there is no space to show them all. The calculated ESI-MS [M+H]+ spectra for each are shown in FIGS. 19(b) and (c). [M−H]− spectra were also calculated but are not shown here, though they are inputs to the autoencoder.

While a deep-learning autoencoder would have difficulty relating the SMILES structures directly to the SIMS spectrum shown in FIG. 2, it has more success in relating the in silico generated ESI-MS spectra to the SIMS spectra of a UV and/or ozone exposed sample. This issue is discussed in more depth in the next section.

### Feasibility of Training Data Sets

A final comment regarding the feasibility of recording training sets by SIMS is justified. Deep learning is a powerful tool, but it can be impractical if the training set of data required is too large and expensive to record. I feel it is worthwhile mentioning here some estimates (though they are frankly no more than guesses, because to make them more quantitative would be extremely expensive in SIMS instrument time) of how large those training sets would need to be to produce a useful trained network for SIMS practitioners to perform automated “spectrum-to-structure” analyses. This is another way of looking at the reasoning behind the choices described in the invention above.

If we were to train a network to relate SMILES structures directly with SIMS spectra, I would estimate this would need about 1,000,000 spectra of different polymers and molecules in the training set, assuming it is possible at all. This is far too expensive to be practical. In the entire lifetime of a SIMS instrument fewer than 1,000,000 spectra are recorded for different samples, so that the instrument would be obsolete or scrapped before the calibration data was complete. Even if sample preparation cost only $10 per sample, this would mean $10,000,000 would be spent on sample preparation, which is impractical.

If we consider using the UV and/or ozone and/or hydrogen sequences of spectra, I'd expect that this reduces the size of the training data set needed to spectrum sequences for about 1,000 reference specimens, because many of the ambiguities present are resolved by this chemical technique and do not need to be learned by the network. So, again assuming it is possible at all, calibrating a SIMS/neural network system in this way is probably expensive but likely feasible, especially if the training set could be judiciously divided into one common to all instruments and one that had to be acquired on the particular instrument in question.

If we now add the use of in silico mass spectrum simulation at plural ionization or fragmentation energies, whether for ESI-MS or some other, and using an autoencoder to link this with SIMS spectra, I would estimate that the size of the training set needed falls again, to between around 30 to 100. This becomes feasible to mount on a single sample holder as a set of reference specimens (some of which are polymer films, for example). This means that it becomes very practical and inexpensive to calibrate an instrument. Certainly the instrument could acquire the reference data for the training set in less than a day, for one of its ion guns in one ion gun condition (beam current, primary ion energy, cluster distribution). There may be two or three such ion beam conditions that the user intends to apply quantitatively in future work, so that the data set for training would certainly take less than a week. Therefore the methods set out above may make automated spectrum-to-structure interpretation of SIMS spectra practical for the first time, whereas previously it would have been impossible or prohibitively expensive.

### FIGURE CAPTIONS

FIG. 1: Schematic of a typical SIMS experiment in a time-of-flight (ToF-SIMS) instrument, comprising a primary ion gun (100), pulsing optics (110), focusing optics (120), raster deflection plates (130), sample under analysis (140), extractor (150), transport optics (160), ion mirror (170), detector (180) and electron flood gun for charge neutralisation (190).
FIG. 2: Typical SIMS spectrum resulting from a complex organic molecule (in this case morphine). Abundance is a measure of the number of counts registered for each mass-to-charge ratio value.
FIG. 3: Line drawing of the external appearance of a typical commercial SIMS instrument, with visible components including the reflectron analyser (310), ion guns (320 and 330), entry lock (340), viewport (350) giving optical access to the analyser chamber, and transfer arm (360) facilitating sample transfer to and from the analyser chamber.
FIG. 4: UV light sources, (a) a typical UV (or UV+ozone) producing lamp. The model shown is type GTL3, with an E17 screw-type connection and 3 W electrical dissipation, and (b) a typical UV producing light-emitting diode unit.
FIG. 2: View of one type of commercial sample holder (510) and the disc-shaped sample “stub” (520) that fits into it.
FIG. 6: Schematic diagram of one embodiment of the UV/ozone exposure apparatus in this invention, showing UV lamps (640) high-wavelength-pass filter (610) and electronic ballast (620) designed to control the UV lamp current during start-up.
FIG. 7: Schematic diagram showing how oxidative species are created and react with the specimen (substrate) surface during UV/ozone cleaning. Similar species are present in one embodiment of the invention, but not for cleaning, instead for an entirely different purpose: to help elucidate the composition of a specimen.
FIG. 8: shows a schematic vertical cross-section through a typical commercial SIMS instrument. There is an analysis chamber (1500) nominally at ultra-high vacuum (UHV), with magnetic sector analyser (1510). Pumps (1505) maintain vacuum in the various parts of the system. Valves (1520) are opened and closed to allow a sample into the analytical chamber from the entry lock (1525). A transfer arm (1535) is used to move the sample between said analysis chamber and said entry lock. When withdrawing a sample from the system the entry lock is brought back up to atmospheric pressure by admitting gas (typically nitrogen) from cylinder (1515). The said entry lock typically has a transparent glass window (1530).
FIG. 9: One configuration of the present experiment in which the sample is moved, in air, from SIMS system to an enclosure (1610) containing the UV/ozone producing lamps (1620).
FIG. 10: Another configuration of the present invention, in which the enclosure containing UV/ozone producing lamps (1720) is integrated with the SIMS system entry lock (1710). This requires a UV-transparent window on said entry lock and the back-fill gas cylinder (1700) to contain oxygen or an oxygen-containing gas mixture (e.g. dry air).
FIG. 11: One embodiment of hydrogen production apparatus. In (a) the switch is open and no hydrogen is being produced by the button cells, 720, or passing through the sealed palladium/palladium alloy tube 730. Under the control of the programmable logic controller, 750, if the switch is closed (as shown in (b)) a current flows through the cells determined in advance by the value of resistor R, causing hydrogen production. Hydrogen permeates through the said sealed Pd/Pd alloy tube.
FIG. 12: One possible simple embodiment B of the cell enclosure.
FIG. 13: Schematic of one possible embodiment of the cell-enclosure type B (incorporating a pressure relief valve). The labelled “inside enclosure” space is the sample enclosure, whereas 930 is the cell-enclosure. In this figure (a) dormant and (b) hydrogen-producing states are shown.
FIG. 14: Schematic diagram showing the near-impact zone of an incident primary ion in SIMS. Note the high energy dissipated close to the impact, and the lower energy region further from the impact, resulting in small fragments close to the impact, and larger fragments further away from it.
FIG. 15: Training of the Autoencoder for spectrum recognition of progressive UV and/or ozone exposure.
FIG. 16: Application of the trained network to SIMS spectra from an unknown analyte (comparison with a putative structure M for this analyte).
FIG. 17: Octave code for simulated ozonolysis of double carbon-carbon bonds in an arbitrary trial molecule.
FIG. 18: Octave code for simulated ozonolysis of triple carbon-carbon bonds in an arbitrary trial molecule
FIG. 19: Example calculated [M+H]− ESI-MS spectra from (a) morphine, and ozonolysis derivatives of morphine (b) and (c). In each case three spectra are plotted, for 10 eV fragmentation energy (x), 20 eV fragmentation energy (+) and 40 eV fragmentation energy (filled circle). The information contained in these spectra for increasing fragmentation energy are useful in relating to SIMS spectra that contain a range of fragmentation energies, as shown in FIG. 14.

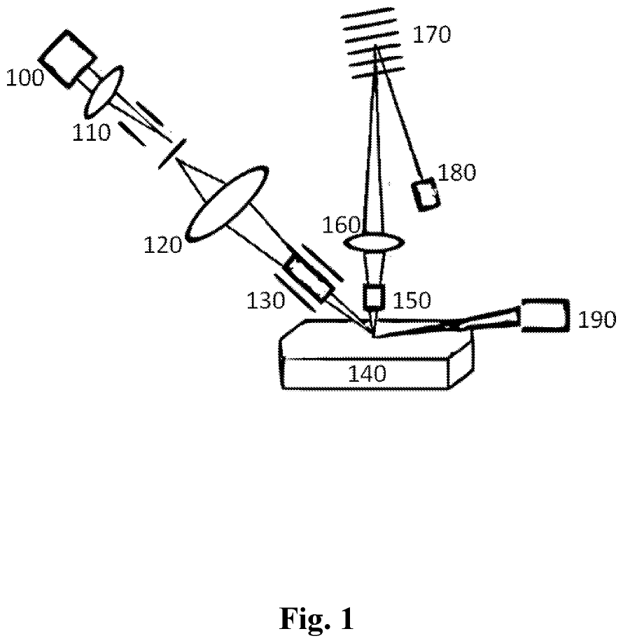

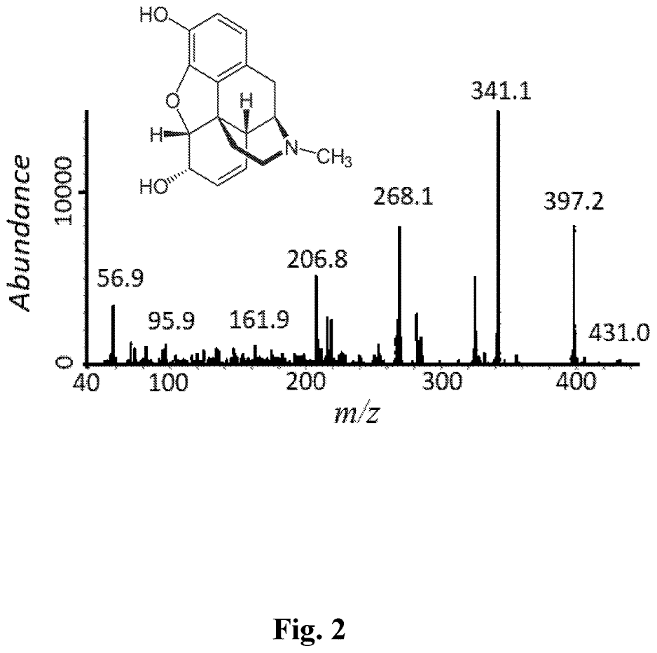

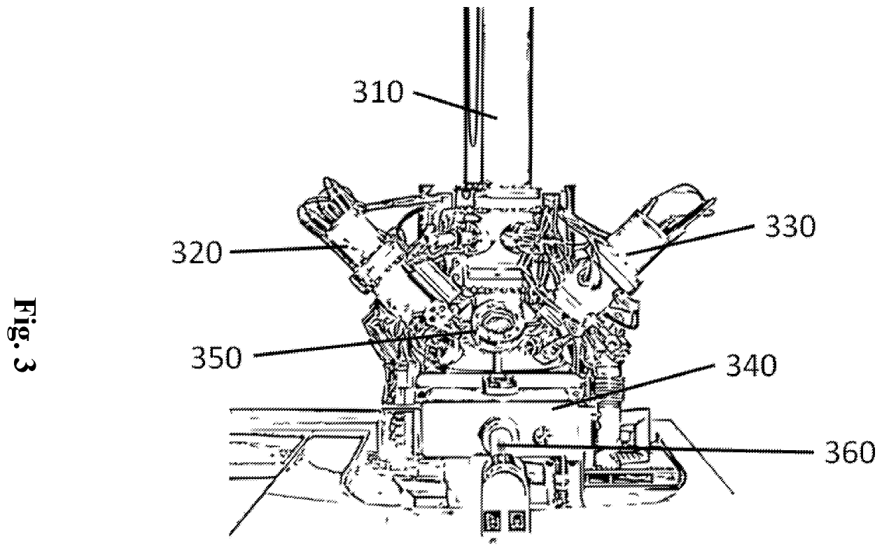

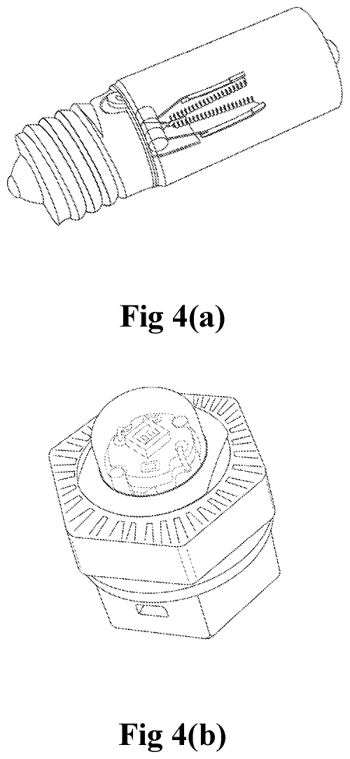

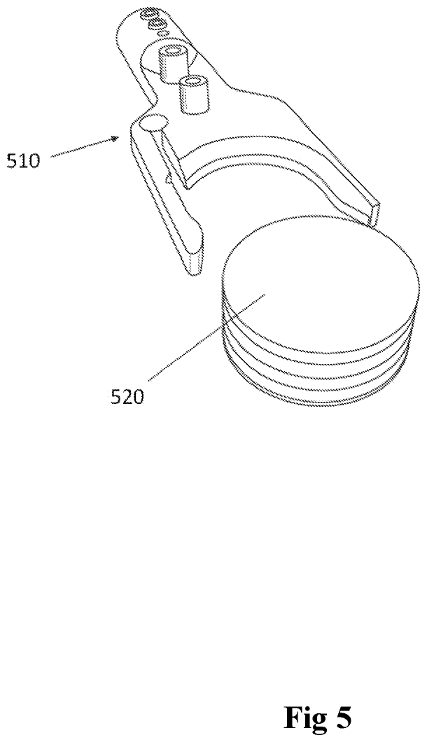

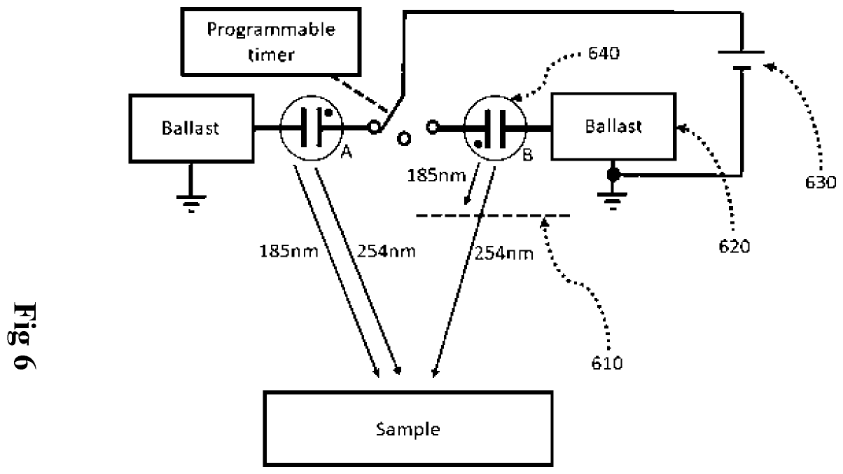

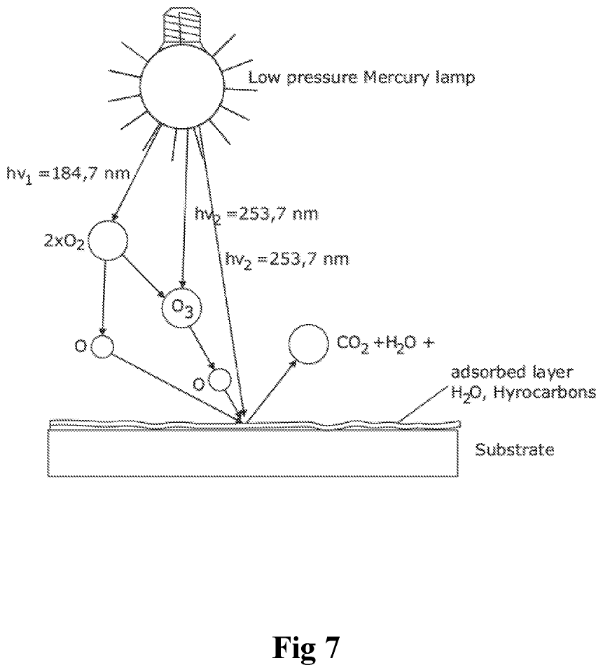

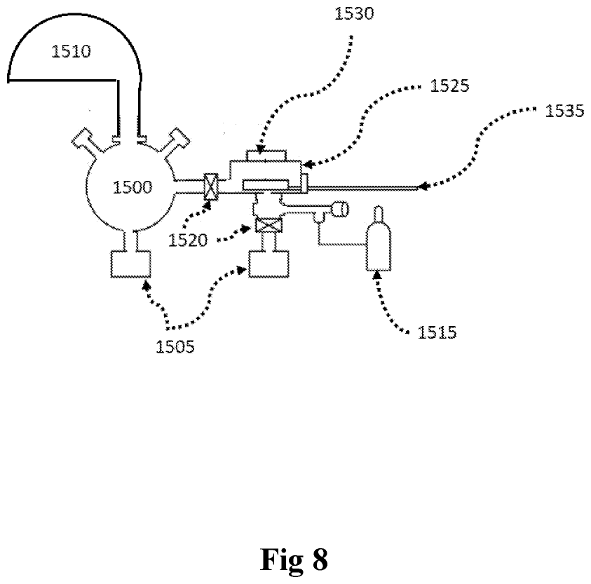

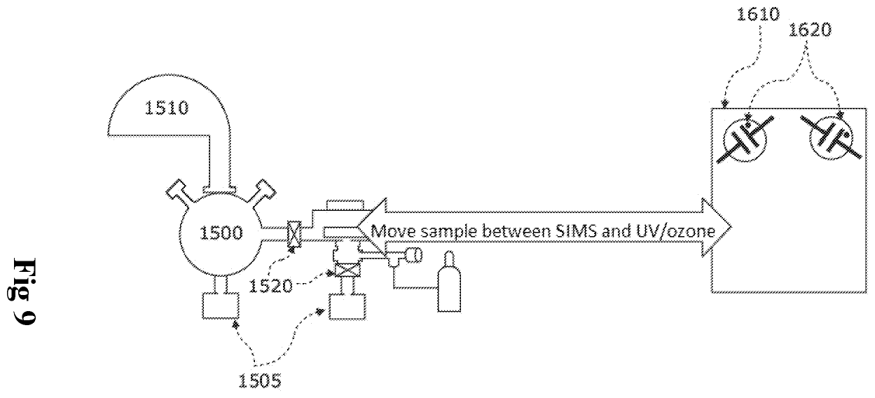

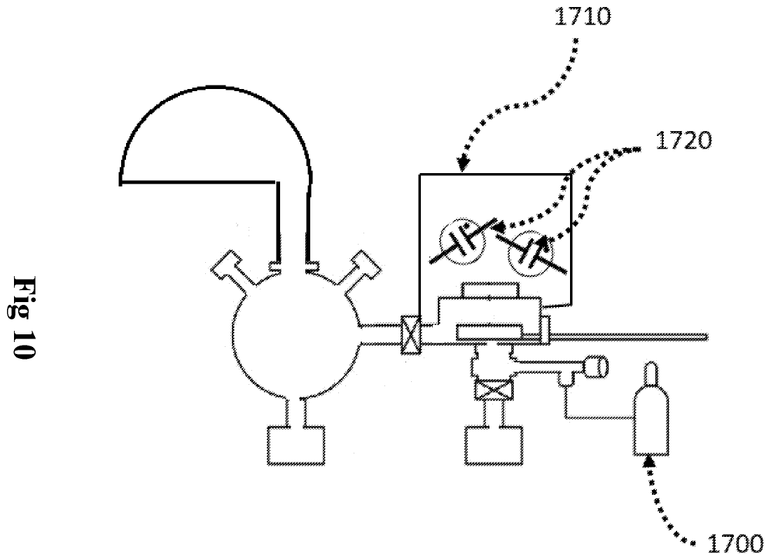

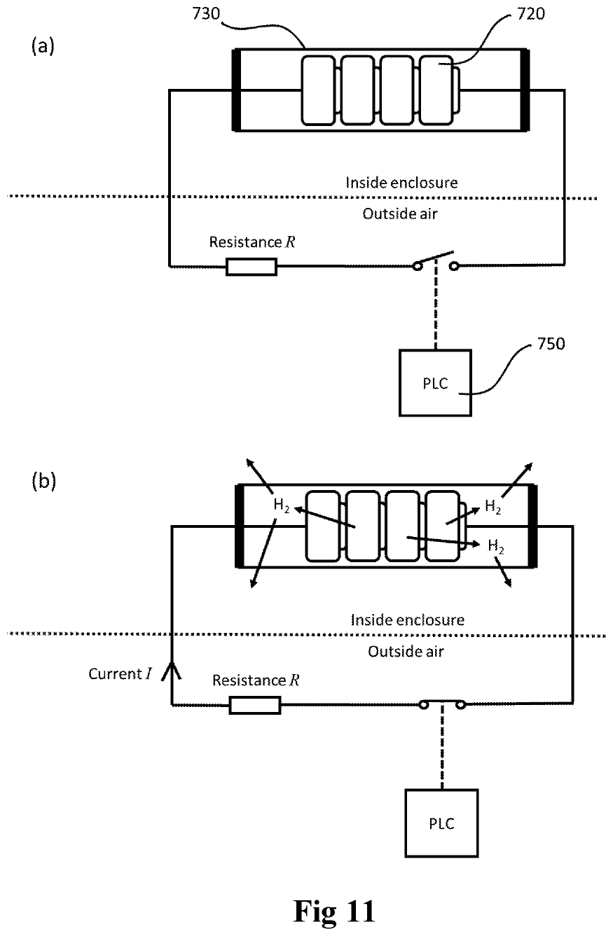

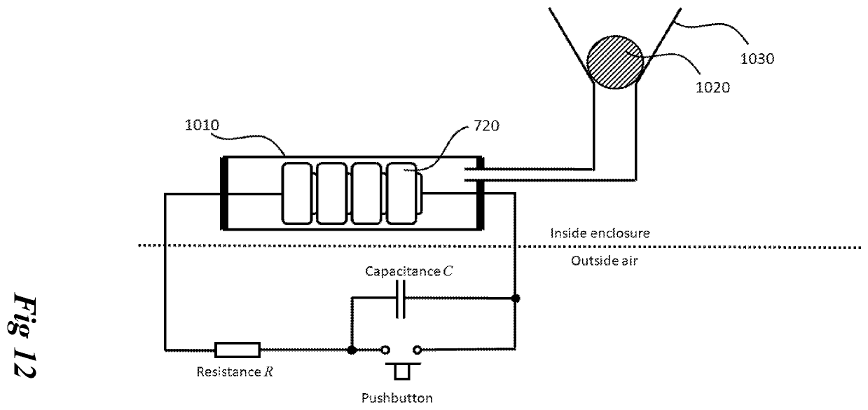

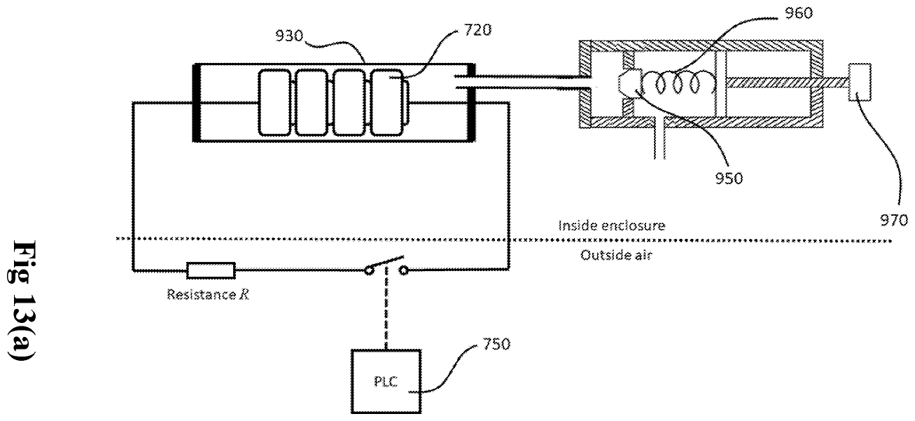

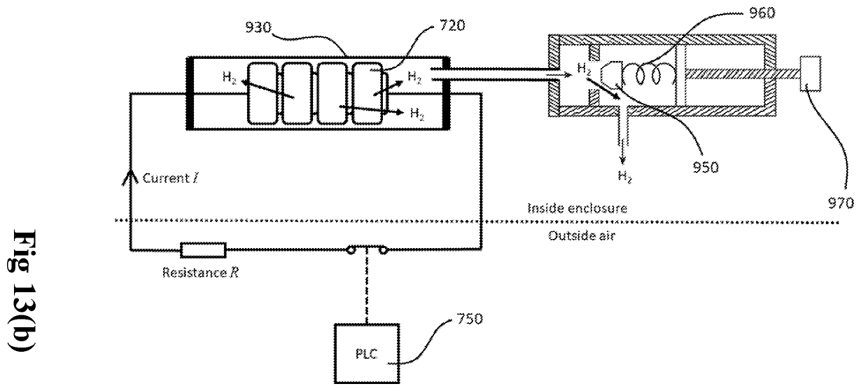

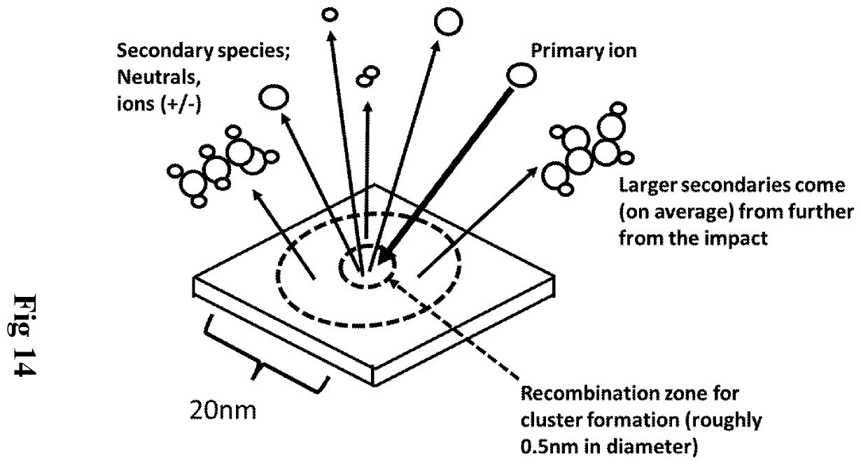

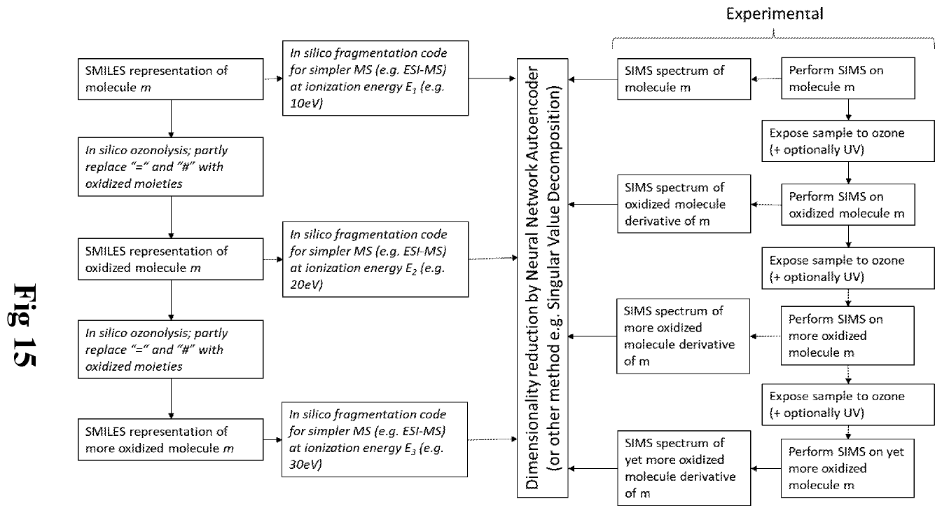

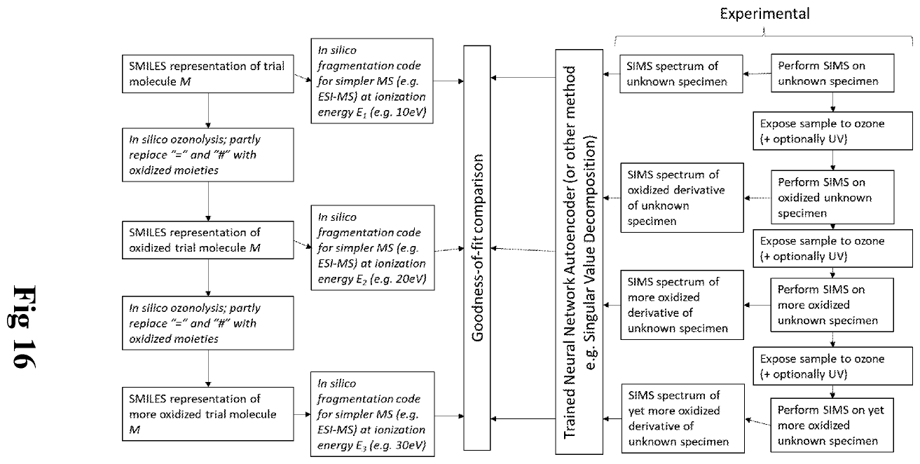

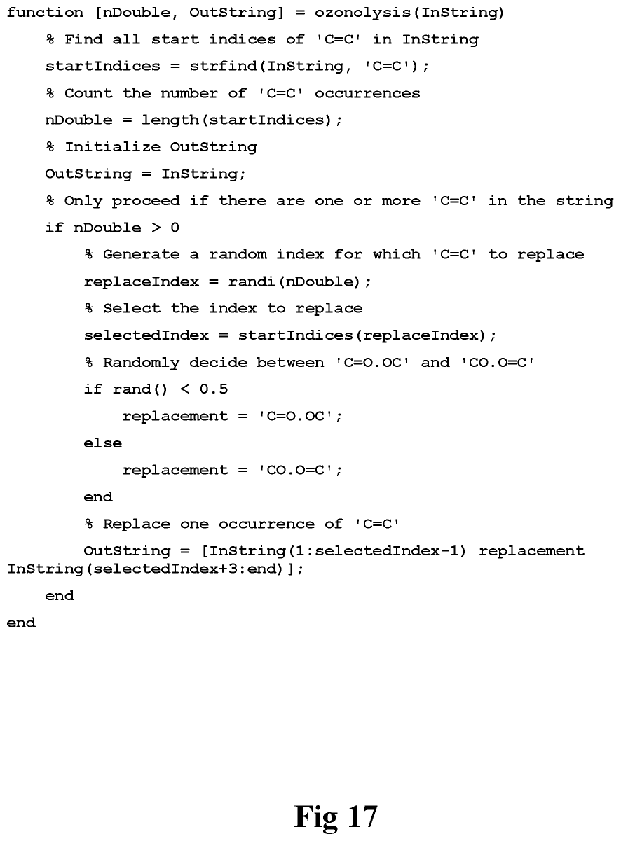

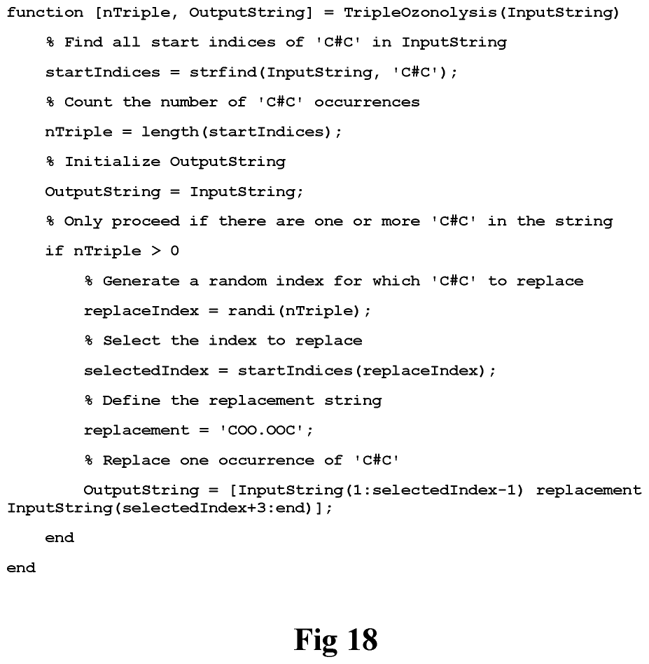

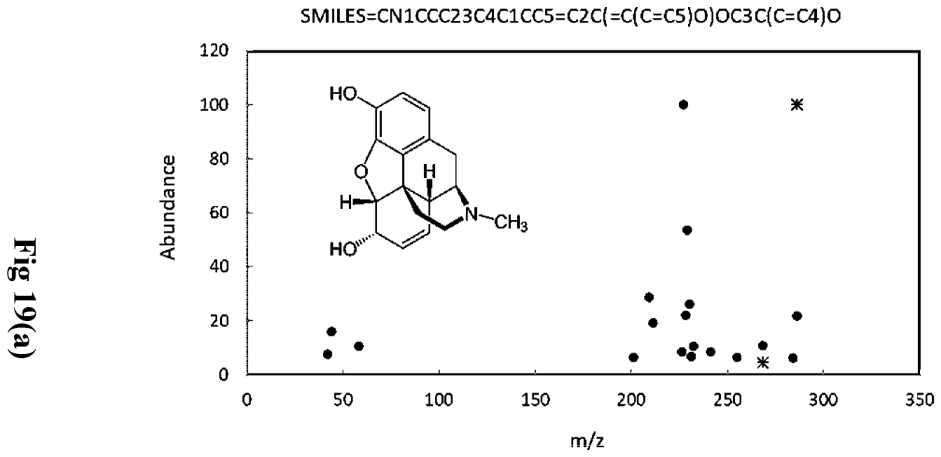

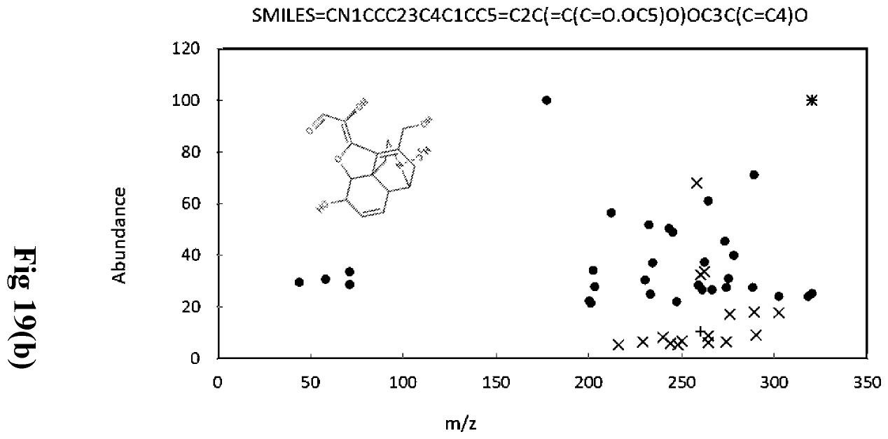

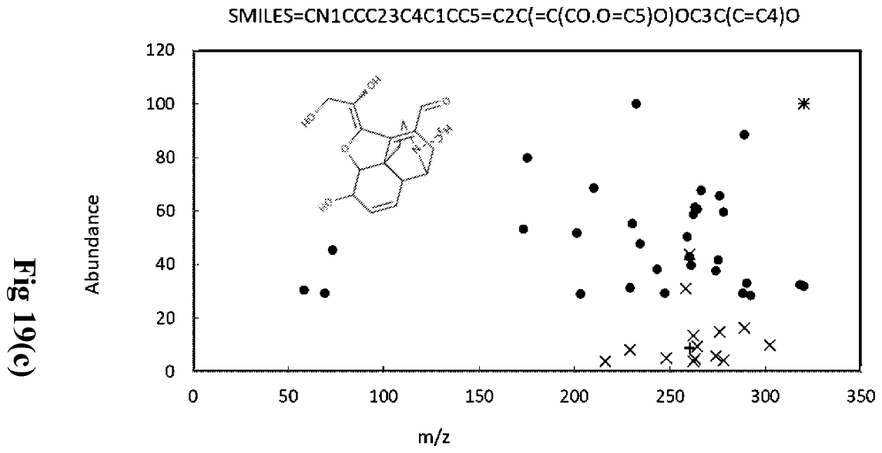

### CITATION LIST

¹Paul van der Heide, Secondary Ion Mass Spectrometry: An Introduction to Principles and Practices, Wiley, Chichester, ISBN: 978-1-118-48048-9, September 2014
²Peter Williams, Secondary Ion Mass Spectrometry, Annual Review of Materials Science, Vol. 15, pp 517-548 (August 1985) https://doi.org/10.1146/annurev.ms.15.080185.002505
³S G Boxer, M L Kraft and P K Weber, Advances in Imaging Secondary Ion Mass Spectrometry for Biological Samples, Annual Review of Biophysics, Vol. 38, pp 53-74 (June 2009) https://doi.org/10.1146/annurev.biophys.050708.133634
⁴Determining Double Bond Position in Lipids Using Online Ozonolysis Coupled to Liquid Chromatography and Ion Mobility-Mass Spectrometry, Rachel A. Harris, Jody C. May, Craig A. Stinson, Yu Xia, and John A. McLean, Analytical Chemistry 2018 90 (3), 1915-1924, DOI: 10.1021/acs.analchem.7b04007
⁵Online Ozonolysis Combined with Ion Mobility-Mass Spectrometry Provides a New Platform for Lipid Isomer Analyses, Berwyck L. J. Poad, Xueyun Zheng, Todd W. Mitchell, Richard D. Smith, Erin S. Baker, and Stephen J. Blanksby, Analytical Chemistry 2018 90 (2), 1292-1300
DOI: 10.1021/acs.analchem.7b04091
⁶Determining Fingerprint Age with Mass Spectrometry Imaging via Ozonolysis of Triacylglycerols, Paige Hinners, Madison Thomas, and Young Jin Lee., Analytical Chemistry 2020 92 (4), 3125-3132, DOI: 10.1021/acs.analchem.9b04765
⁷Angelo, M., Bendall, S., Finck, R. et al. Multiplexed ion beam imaging of human breast tumors. Nat Med 20, 436-442 (2014). https://doi.org/10.1038/nm.3488
   ⁸Agüi-Gonzalez, P.; Dankovich, T. M.; Rizzoli, S. O.; Phan, N. T. N. Gold-Conjugated Nanobodies for Targeted Imaging Using High-Resolution Secondary Ion Mass Spectrometry. Nanomaterials 2021, 11, 1797. https://doi.org/10.3390/nano11071797
   ⁹G.-Q. Jin, D.-e. Sun, X. Xia, Z.-F. Jiang, B. Cheng, Y. Ning, F. Wang, Y. Zhao, X. Chen, J.-L. Zhang, Angew. Chem. Int. Ed. 2022, 61, e202208707; Angew. Chem. 2022, 134, e202208707.
   ¹⁰Roel De Mondt, Luc Van Vaeck, Jens Lenaerts, In Situ Temperature-Time Effect on MetA-S-SIMS, Journal of the American Society for Mass Spectrometry, Volume 18, Issue 3,2007, Pages 382-384, https://doi.org/10.1016/j.jasms.2006.09.029.
   ¹¹D Léonard, B Keller, Y Chevolot, M Wieland, H. J Mathieu,
Round robin of time-of-flight secondary ion mass spectrometry damage studies of a photoimmobilized reagent on diamond surfaces designed for surface glycoengineering, Applied Surface Science, Volumes 144-145, 1999, Pages 409-413, https://doi.org/10.1016/S0169-4332(98)00833-2.
¹²I. S. Gilmore and M. P. Seah, Static SIMS: towards unfragmented mass spectra—the G-SIMS procedure, Applied Surface Science 161 (2000) 465-480.
¹³I S Gilmore and M P Seah, Organic molecule characterization—G-SIMS, Applied Surface Science 231-232 (2004) 224-229.
¹⁴I S Gilmore and M P Seah, G_SIMS of crystallisable organics, Applied Surface Science 203-204 (2003) 551-555
¹⁵Felicia M. Green, Felix Kollmer, Ewald Niehuis, Ian S. Gilmore and Martin P. Seah, Imaging G-SIMS: a novel bismuth-manganese source emitter, Rapid Commun. Mass Spectrom. 2008; 22: 2602-2608
¹⁶F. M. Green, I. S. Gilmore, and M. P. Seah, Molecular Structure and Identification Through G-SIMS and SMILES, Surface Analysis and Techniques in Biology, Ed Vincent S. Smentkowski, Springer, Heidelberg, 2014
¹⁷Jumper, J., Evans, R., Pritzel, A. et al. Highly accurate protein structure prediction with AlphaFold. Nature 596, 583-589 (2021). https://doi.org/10.1038/s41586-021-03819-2
¹⁸Waymouth, John (1971). Electric Discharge Lamps. Cambridge, MA: The M.I.T. Press. ISBN 978-0-262-23048-3.
¹⁹Waymouth, John (1971). Electric Discharge Lamps. Cambridge, MA: The M.I.T. Press. ISBN 978-0-262-23048-3.
²⁰H Amandusson, L.-G Ekedahl, H Dannetun, Hydrogen permeation through surface modified Pd and PdAg membranes, Journal of Membrane Science, Volume 193, Issue 1, 2001, Pages 35-47, ISSN 0376-7388, https://doi.org/10.1016/S0376-7388(01)00414-8.
²¹A. G. Knapton, Palladium Alloys for Hydrogen Diffusion Membranes, Platinum Metals Rev., 1977, 21, (2) p 44-50
²²Jae Seok Park et al., “Development of small and efficient ozone generators using corona discharge,” 5th Korea-Russia International Symposium on Science and Technology. Proceedings. KORUS 2001 (Cat. No. 01EX478), Tomsk, Russia, 2001, pp. 282-284 vol. 1, doi: 10.1109/KORUS.2001.975124.
²³John R. Vig, “UV/ozone cleaning of surfaces”, Journal of Vacuum Science & Technology A 3, 1027-1034 (1985) https://doi.org/10.1116/1.573115
²⁴W. L. BAUN, ISS/SIMS CHARACTERIZATION OF UV/03 CLEANED SURFACES, Applications of Surface Science 6 (1980) 39-46
²⁵S V Roberson, A J Fahey, A Sehgal and A Karim, Multifunctional ToF-SIMS: combinatorial mapping of gradient energy substrates, Applied Surface Science 200 (2002) 150-164
²⁶L. A. Zazzera, XPS and SIMS Study of Anhydrous HF and UV/Ozone-Modified Silicon (100) Surfaces, J. Electrochem. Soc., Vol. 136, No. 2, February 1989
²⁷J. Shao, C. M. Carr, C. P. Rowlands & J. Walton (1999) XPS, SIMS, and ESR Studies of UV/Ozone-irradiated Silk and Wool, Journal of the Textile Institute, 90:4, 459-468, DOI: 10.1080/00405000.1999.10750045.
²⁸Shangwei Huang et al 2020 J. Electrochem. Soc. 167 090538
²⁹Jeong, B. J.; Jo, Y. N. A Study on the Self-Discharge Behavior of Zinc-Air Batteries with CuO Additives. Appl. Sci. 2021, 11, 11675.
https://doi.org/10.3390/appl 12411675
³⁰For example, Varta type V 150 H2 MF, see https://www.varta-ag.com/en/industry/product-solutions/hydrogen
³¹Determining Double Bond Position in Lipids Using Online Ozonolysis Coupled to Liquid Chromatography and Ion Mobility-Mass Spectrometry, Rachel A. Harris, Jody C. May, Craig A. Stinson, Yu Xia, and John A. McLean, Analytical Chemistry 2018 90 (3), 1915-1924, DOI: 10.1021/acs.analchem.7b04007
³²Online Ozonolysis Combined with Ion Mobility-Mass Spectrometry Provides a New Platform for Lipid Isomer Analyses, Berwyck L. J. Poad, Xueyun Zheng, Todd W. Mitchell, Richard D. Smith, Erin S. Baker, and Stephen J. Blanksby, Analytical Chemistry 2018 90 (2), 1292-1300
DOI: 10.1021/acs.analchem.7b04091
³³Determining Fingerprint Age with Mass Spectrometry Imaging via Ozonolysis of Triacylglycerols, Paige Hinners, Madison Thomas, and Young Jin Lee., Analytical Chemistry 2020 92 (4), 3125-3132, DOI: 10.1021/acs.analchem.9b04765
³⁴Qi, Y., Hu, D., Jiang, Y., Wu, Z., Zheng, M., Chen, E. X., Liang, Y., Sadi, M. A., Zhang, K., Chen, Y. P., Recent Progresses in Machine Learning Assisted Raman Spectroscopy. Adv. Optical Mater. 2023, 11, 2203104.
https://doi.org/10.1002/adom.202203104.
³⁵Ho C S, Lam C W, Chan M H, Cheung R C, Law L K, Lit L C, Ng K F, Suen M W, Tai H L. Electrospray ionisation mass spectrometry: principles and clinical applications. Clin Biochem Rev. 2003; 24(1):3-12. PMID: 18568044; PMCID: PMC1853331.
³⁶Wang F, Liigand J, Tian S, Arndt D, Greiner R, Wishart D S. CFM-ID 4.0: More Accurate ESI-MS/MS Spectral Prediction and Compound Identification. Anal Chem. 2021 Aug. 31; 93(34):11692-11700. doi: 10.1021/acs.analchem.1c01465. Epub 2021 Aug. 17. PMID: 34403256; PMCID: PMC9064193.
³⁷Weininger D (February 1988). “SMILES, a chemical language and information system. 1. Introduction to methodology and encoding rules”. Journal of Chemical Information and Computer Sciences. 28 (1): 31-6. doi:10.1021/ci00057a005.
³⁸Weininger D, Weininger A, Weininger J L (May 1989). “SMILES. 2. Algorithm for generation of unique SMILES notation”. Journal of Chemical Information and Modeling. 29 (2): 97-101. doi:10.1021/ci00062a008.
³⁹Weininger D (August 1990). “SMILES. 3. DEPICT. Graphical depiction of chemical structures”. Journal of Chemical Information and Modeling. 30 (3): 237-43. doi:10.1021/ci00067a005
⁴⁰Wang F, Liigand J, Tian S, Arndt D, Greiner R, Wishart D S. CFM-ID 4.0: More Accurate ESI-MS/MS Spectral Prediction and Compound Identification. Anal Chem. 2021 Aug. 31; 93(34):11692-11700. doi: 10.1021/acs.analchem.1c01465. Epub 2021 Aug. 17. PMID: 34403256; PMCID: PMC9064193.
⁴¹Ozone Production by Corona Discharges, September 2002 Journal of the IEST 45(1):98-105, DOI:10.17764/jiet.45.1.p5643235m507702u
⁴²Bashari Rad, Babak & Bhatti, Harrison & Ahmadi, Mohammad. (2017). An Introduction to Docker and Analysis of its Performance. IJCSNS International Journal of Computer Science and Network Security. 173. 8.
⁴³Wang F, Liigand J, Tian S, Arndt D, Greiner R, and Wishart D. (2021) CFM-ID 4.0: More Accurate ESI MS/MS Spectral Prediction and Compound Identification. Anal Chem. 93(34):11692-11700.

1. A process for producing secondary ion mass spectrometry spectra of a sample comprising the steps of: producing a plurality of different oxidation states of the sample in a surface thereof by exposing the sample surface to an agent configured to change the oxidation state of said sample surface; placing the sample in a secondary ion mass spectrometry apparatus; obtaining an secondary ion mass spectrum for each of the plurality of oxidation states of the said sample surface; identifying materials within the sample by analysing the plurality of spectra.

2. A process according to claim 1, wherein the sample is exposed to the agent configured to change the oxidation state of the said surface of the sample a plurality of times sequentially, wherein in each subsequent exposure of the sample to the agent, the oxidation state of the surface of the sample is changed relative to the oxidation state of the sample surface resulting from the preceding exposure to the agent configured to change the oxidation state of the said sample surface.

3. A process according to claim 1, wherein the sample is divided into a plurality of sub-samples each having a sub-sample surface, and wherein a different oxidation state of the sub-sample surface is produced for each sub-sample.

4. A process according to any preceding claim, wherein the agent configured to change the oxidation state of the sample surface is a gaseous agent.

5. A process according to any preceding claim, wherein the agent configured to change the oxidation state of the sample surface includes one or more of: ultraviolet light, ozone and hydrogen.

6. A process according to claim 5, wherein ultraviolet light is provided by at least one ultraviolet (UV) lamp, wherein UV light emitted from the at least one UV lamp is directed at said sample surface.

7. A process according to claim 6, wherein the UV light emitted from the at least one UV lamp is in the wavelength range 200 nm to 300 nm.

8. A process according to claim 6 or 7, wherein the UV lamp is a mercury vapour lamp.

9. A process according to claim 5, wherein ozone is provided by an ozone-producing device producing ozone gas at concentration in the range 0.01 to 20 parts-per-million in the gas around the said specimen.

10. A process according to any preceding claim, including the step of controlling the degree of change of the oxidation state of said sample surface by controlling one or more of: the time of exposure of the said sample surface to the agent; the concentration of the agent; and the wavelength and/or frequency of the agent.

11. A process according to any preceding claim, wherein the step of identifying materials within the sample by analysing the plurality of spectra includes performing multivariate analysis.

12. A process according to any preceding claim, wherein the step of identifying materials within the sample by analysing the plurality of spectra includes performing simulated molecular fragmentation and relating this fragmentation to the spectra by an artificial neural network.

13. A device for capturing secondary ion mass spectrometry spectra configured to perform the process of any of claims 1 to 12, comprising: a sample holder; a source of the agent configured to change the oxidation state of a surface of a sample held in the sample holder; means to control exposure of the sample surface to the agent configured to change the oxidation state of said surface; and a secondary ion mass spectrometer capable of recording a plurality of secondary ion mass spectrometry spectra one for each oxidation state of the sample surface.

14. A device according to claim 13, further comprising a data processor configured to perform simulated molecular fragmentation and relate this fragmentation to the spectra by a neural network.

15. A device according to claim 13 or 14, wherein the sample holder is contained in an enclosure.

16. A device according to any of claims 13 to 15, wherein the agent configured to change the oxidation state of the sample surface is a gaseous agent.

17. A device according to any of claims 13 to 16, wherein the source of the agent configured to change the oxidation state of the sample surface is one or more of: ultraviolet light, ozone and hydrogen.

18. A device according to claim 17, wherein the ultraviolet light is provided by at least one ultraviolet (UV) lamp, wherein UV light emitted from the at least one UV lamp is directed at said sample surface.

19. A device according to claim 18, wherein the UV light emitted from the at least one UV lamp is in the wavelength range 200 nm to 300 nm.

20. A device according to claim 18 or 19, wherein the at least one UV lamp is mercury vapour lamp.

21. A device according to any of claims 17 to 20, further comprising an ozone generator configured to release ozone around a sample situated in the sample holder.

22. A device according to claim 21 wherein the ozone generator is the at least one UV lamp emitting in the region 100-300 nm in the air around the sample.

23. A device according to claim 22, wherein the at least one UV lamp emits UV at 185 nm and/or 254 nm

24. A device according to 17 to 20, further comprising a hydrogen source configured to release hydrogen around the sample in the sample holder.

25. A device according to claim 24, wherein the hydrogen source is at least one zinc air cell.

26. A device according to any of claims 13 to 25, wherein the sample holder is adapted to hold a plurality of sub-samples, each sub-sample having a surface with a different oxidation state, and wherein the secondary ion mass spectrometry instrument is configured to record secondary ion mass spectrometry spectra for each of the sub-samples.
